(19) United States
(12) Patent Application Publication
(10) Pub. No.: US 2013/0064781 A1
(43) Pub. Date: Mar. 14, 2013

(54) METHOD FOR MASKING THE BITTERNESS OF A UV-SCREENING AGENT

(71) Applicants: Karine CHEVET, CHATEAUNEUF SUR LOIRE (FR); Valerie ALARD, ORLEANS (FR); Eric PERRIER, LES COTES d'AREY (FR); Marie-Laure SOUVIE, SEMOY (FR)

(72) Inventors: Karine CHEVET, CHATEAUNEUF SUR LOIRE (FR); Valerie ALARD, ORLEANS (FR); Eric PERRIER, LES COTES d'AREY (FR); Marie-Laure SOUVIE, SEMOY (FR)

(73) Assignee: LVMH RECHERCHE, SAINT JEAN DE BRAYE (FR)

(21) Appl. No.: 13/599,703

(22) Filed: Aug. 30, 2012

(30) Foreign Application Priority Data
Sep. 9, 2011 (FR) 1158048

(51) Int. Cl. A61K 8/60; A61K 8/97; A61K 8/49; A61Q 17/04; A61K 8/35

(52) U.S. Cl. 424/60; 424/59

(57) ABSTRACT

The subject of the invention is the use of a sweetener such as sucralose or an extract of Stevia, for masking the bitterness of hydrophilic organic UV-screening agents in cosmetic or dermatological compositions.

The sweetener may advantageously be used in combination with a mint aroma or a salt, for example sodium chloride. This mixture of sweetener with a second gustatory agent makes it possible to mask the bitterness of the hydrophilic UV-screening agent without changing the nature of the fragrance or the colour of the composition.

The invention relates to a method for masking the bitterness of a hydrophilic organic UV-screening agent in a facial cosmetic or dermatological composition.

### PRIOR ART AND PURPOSES OF THE INVENTION

The incorporation of sweeteners into lipsticks or lip glosses in order to give them a sweet flavour has already been proposed. In this case, the amount of sweetener is generally very low, about from 0.01 to 0.02% by weight of the weight of the composition. Makeup products for the lips are oil- and wax-based and contain lipophilic ingredients. Thus, the organic UV-screening agents chosen to be incorporated into lipsticks or lip glosses are lipophilic.

In order to apply a care cream, a liquid foundation or a suncream to the face, the user generally takes a sufficient amount of cream in the hand in order to subsequently spread it on the facial skin and to promote its penetration by rubbing. A generous application of the cosmetic product can result in the product unintentionally spreading over onto the lips.

It has, however, been observed that some of these facial care or makeup products give a very bitter taste when they come into contact with the area around the lips during their application to the whole of the face.

The principal purpose of the invention is to solve the technical problem consisting of the provision of a cosmetic or dermatological composition intended to be applied to the skin which does not give any sensation of bitterness in the event of unexpected contact with the lips.

It has been discovered more precisely that, in a facial care cream containing an aqueous phase, oils, surfactants, fragrances, hydrophilic organic UV-screening agents and lipophilic organic UV-screening agents in conventional proportions, only the hydrophilic organic UV-screening agents cause a bitter taste. The same care cream containing lipophilic organic UV-screening agents but devoid of hydrophilic organic UV-screening agent is not at all bitter. The Applicant has demonstrated that lipophilic organic UV-screening agents, in particular ethylhexylmethoxy cinnamate, butylmethoxydibenzoylmethane, bis-ethylhexyloxyphenol methoxyphenyl triazine, and octocrylene do not generate any bitterness when they are formulated in the fatty phase of a cosmetic composition.

The present invention therefore proposes a cosmetic or dermatological composition intended to be applied to the skin, in which the bitterness of a hydrophilic organic UV-screening agent is masked. The Applicant has found that it is possible to mask the bitterness of this type of screening agent by incorporating into the composition a sufficient amount of a sweetener, without this amount disrupting the initial cosmetic properties of the product, such as the physicochemical and sensory properties.

### DESCRIPTION OF THE INVENTION

Thus, the subject of the present invention is a method for masking the bitterness of a hydrophilic organic UV-screening agent in a cosmetic composition containing an aqueous phase and in the form of a facial care cream, of a face lotion, of a face serum or fluid, of a foundation, of a milk, of a makeup-removing lotion or of a facial antisun product, said method comprising the addition to said composition:
of a sufficient amount of at least one first gustatory agent chosen from sweeteners, and
of a sufficient amount of at least one second gustatory agent chosen from taste enhancers and aromas.

The term “sufficient amount” corresponds to an amount sufficient to mask the taste of the hydrophilic organic UV-screening agent when the composition which contains same has been applied to the area around the lips or to the lips.

Hydrophilic UV Screening Agent

The hydrophilic organic UV-screening agent may be responsible for the bitterness experienced by the user when the latter accidentally applies the composition to the lips or the area around the lips.

Bitterness is a characteristic which is felt at the back of the tongue and the palate. It can be evaluated by a panel of users who have been trained beforehand in the recognition and quantitative evaluation of the level of bitterness of a compound in such a way that the evaluations of each user are reproducible, and the average measurement of the bitterness of a product measured by this panel is reliable.

The bitterness of the hydrophilic UV-screening agent can be evaluated by placing 0.2 g of an aqueous solution containing 0.1 to 5% by weight thereof on the area around the lips or on the lips of the members of a panel. An aqueous solution of hydrophilic UV-screening agent is considered to be bitter if the average bitterness evaluated by the panel is at least equal to that of 0.2 g of an aqueous solution of quinine at 8 μmol/l.

The bitterness of an aqueous solution of hydrophilic UV-screening agent can be evaluated by a panel of users. Each member of the panel does not have the same sensitivity to bitterness, but must be reproducible. A sufficient number of reproducible members makes it possible to average the inter-individual disparities in such a way as to obtain a significant average evaluation, and an objective quantification of the bitterness.

In order to judge the reproducibility of a member of the panel, said member is proposed several samples blind. Among these samples, two samples of the same composition are introduced. The evaluation of these two samples must be identical or substantially identical. For example, on a grading scale of from 1 to 5, a difference in grade of 1 is tolerated.

The sensation of bitterness generated by the hydrophilic UV-screening agent is masked in the composition at the moment it is applied to the lips, but it is also possible to advantageously obtain a persistence of the masking of the bitterness of the screening agent for at least 10 minutes, or even for at least 15 minutes, after accidental application of the composition to the lips.

The expression “hydrophilic organic UV-screening agent” is intended to mean any organic compound which absorbs ultraviolet (UV) radiation in the wavelengths range of from 280 nm to 400 nm and which can be dissolved in the aqueous phase of a composition, or which can be dispersed therein in colloidal form or in micellar form.

Among the hydrophilic UV-screening agents, use may be made of the following screening agents denoted below by their INCI name or their chemical name:
terephthalylidene dicamphorsulfonic acid (INCI name: terephthalylidene dicamphor sulfonic acid) sold under the name Mexoryl® SX by Chimex,
the bis-benzoazolyl derivatives as described in patents EP 669 323 and U.S. Pat. No. 2,463,264 and more particularly the compound disodium phenyl dibenzimidazole tetrasulfonate sold under the trade name Neo Heliopan® AP by Haarmann and Reimer,
p-aminobenzoic acid (INCI name: PABA) and derivatives thereof, such as 1-(4-aminobenzoate)-1,2,3-propanetriol (INCI name: Glyceryl PABA) and PEG-25 PABA sold under the name Uvinul® P25 by BASF,
2-phenylbenzimidazole-5-sulfonic acid (INCI name: phenyl-benzimidazole sulfonic acid) sold in particular under the trade name Eusolex® 232 by Merck,
triethanolamine salicylate,
3-(4′-sulfobenzylidene)camphor (INCI name: benzylidene camphor sulfonic acid) sold under the name Mexoryl® SL by Chimex,
methylene bis-benzotriazolyl tetramethylbutylphenol (USAN name: bisoctrizole) sold under the reference Tinosorb® M, or Mixxim® BB/100 by Fairmount Chemical;
3-(4′-trimethylammoniumbenzylidene)-1-bornan-2-one methyl sulfate (INCI name: camphor benzalkonium methosulfate) sold under the name Mexoryl SO by Chimex,
benzophenone-4 sold under the trade name “Uvinul MS40” by BASF, benzophenone-5, and benzophenone-9.

It is also possible to use, as hydrophilic organic UV-screening agent, organic UV-screening molecules which are lipophilic in nature (dissolved or dispersed in a nonaqueous liquid) which have been made hydrophilic by adsorption onto a hydrophilic support of small particle size, for instance polymer particles. Mention may, for example, be made of bisethylhexyloxyphenol methoxyphenyl triazine, which is a lipophilic UV-screening agent adsorbed onto particles of polymethyl methacrylate (PMMA). The hydrophilic organic UV-screening agent may therefore be a lipophilic organic UV-screening molecule adsorbed or absorbed onto a hydrophilic support, which may not screen out UV rays, such as an organic polymer.

The hydrophilic organic UV-screening agent is present in the composition used in the context of the method according to the invention in proportions ranging from 0.05 to 10% by weight relative to the total weight of the composition, preferably ranging from 0.1 to 8% by weight, and better still from 0.1 to 5% by weight relative to the total weight of the composition.

First Gustatory Agent: a Sweetener

The composition comprises a first gustatory agent consisting of a sweetener, which is a compound that has a sweet taste.

The sweetener is advantageously incorporated in an amount sufficient to mask the bitterness of the hydrophilic organic UV-screening agent, without however destabilising the composition or giving too sweet a taste that would become sickly. It is thus preferred for the amount of sweetener to be less than 1% by weight relative to the weight of the composition.

The first sweetening gustatory agent preferably represents from 0.05 to 2% by weight, more preferably from 0.1 to 1% by weight, preferentially from 0.2 to 0.6% by weight, more preferentially from 0.4 to 0.5% by weight, of the weight of the composition.

The sweetener is advantageously water-soluble and can be chosen from the group consisting of the following compounds: sucrose, glucose, fructose, acesulfame K, aspartame, cyclamic acid and also its sodium, potassium and calcium salts, isomalt, saccharin and also its sodium, potassium and calcium salts, sucralose, alitame, thaumatin, glycyrrhizic acid and its salts, neohesperidin dihydrochalcone, steviol glucosides, neotame, the aspartame-acesulfame salt, tagatose, polyglycitol syrup, maltitol, lactitol, xylitol and erythritol, and mixtures thereof.

The sweetener preferably has a sweetening power greater than or equal to 50 times, preferably greater than or equal to 100 times, that of sucrose.

According to one advantageous embodiment, the sweetener is sucralose (CAS number 56038-13-2).

According to another embodiment, the sweetener is chosen from steviol glucosides and plant extracts containing them. The sweetener is, for example, chosen from stevioside, steviolbioside, rebaudiosides A, B, C, D and E, dulcosides A and B, and mixtures thereof. The sweetener is preferably rebaudioside A (sometimes called stevioside) or a plant extract containing rebaudioside A.

Rebaudioside A is a heteroside, the aglycone part of which, called steviol, is linked to two oside groups: one glucose unit and one glucose triholoside (IUPAC name: 19-O-beta-glucopyranosyl-13-O-(beta-gluco-pyranosyl(1-2)-beta-glucopyranosyl(1-3))-beta-glucopyranosyl-β-hydroxykaur-16-en-19-oic acid and CAS No. 58543-16-1). Its sweetening power is 250 to 450 times greater than that of sucrose.

Rebaudioside A can be advantageously extracted from Stevia, more precisely from its leaves. The species Stevia rebaudiana for example contains same. Use may be made, in the context of the invention, of an extract of Stevia rebaudiana, such as that sold under the reference Rebaten 97% Sweetener® by the company SEPPIC.

The plants of the Stevia genus grow naturally in Paraguay; they are also cultivated in South America and in Asia. In order to extract rebaudioside A therefrom, the leaves of the plant are dried and reduced to powder, before undergoing aqueous extraction and then purification.

Second Gustatory Agent: Aroma or Taste Enhancer

The composition can advantageously comprise at least one second gustatory agent, which is chosen from taste enhancers and aromas. The second gustatory agent is preferably water-soluble.

The combination of the two gustatory agents allows persistent masking of the bitterness of the bitter UV-screening agent, or even definitive masking of the bitterness of this screening agent in the composition, once it has been applied to the lips.

Taste enhancers are organic substances which, without having their own pronounced flavour, have the property of prolonging the masking of the bitterness of the hydrophilic UV-screening agent over time.

The taste enhancers are in particular chosen from sodium chloride, potassium chloride, zinc chloride, glutamic acid and its sodium, potassium, magnesium, calcium and ammonium salts, guanylic acid and its salts, inosinic acid and its salts, calcium 5′-ribonucleotide, disodium 5′-ribonucleotide, maltol, ethyl maltol, sodium L-aspartame, DL-alanine, glycine and its sodium salt, L-leucine, lysine hydrochloride, and zinc acetate.

In one advantageous embodiment, a salty taste enhancer, in particular sodium chloride, is preferably used.

The aromas used in the context of the invention cause a sensation by retro-olfaction when they are swallowed and are preferably ingredients which have neither a sweet, nor salty, nor acid taste. These ingredients are used to give the composition a taste. The aromas may have the INCI name “Aroma” appearing on the listing of ingredients of the cosmetic product proposed for sale. They are preferably chosen from natural aromas and natural identical aromas.

The aromas may be chosen from citrus fruit aromas, in particular orange, lemon, grapefruit or mandarin aromas; fruit aromas, in particular cherry, peach, pear or passion fruit aromas; gourmand aromas, in particular vanilla, chocolate, caramel aromas or milk aromas; mint aromas; and mixtures thereof.

Preference is given to mint aromas which have the advantage of effectively prolonging the masking of the bitterness at contents lower than the other aromas, so that they modify the initial perfuming of the product only to a lesser extent at these low contents. Menthol or an extract of mint essential oil can in particular be used as mint aroma.

The concentration of the second gustatory agent preferably ranges from 0.0005 to 0.5%, more preferably from 0.01 to 0.3% by weight relative to the weight of the composition.

An aroma is preferably used in an amount such that it is not accompanied by a significant odour that will disrupt the olfactory signature of the composition.

According to one variant, the composition comprises a hydrophilic organic UV-screening agent, at least one steviol glucoside as first gustatory agent, and at least one salt as second gustatory agent, chosen from sodium chloride, potassium chloride and zinc chloride, and mixtures thereof.

According to another variant, the composition comprises at least one hydrophilic organic UV-screening agent, sucralose as first gustatory agent and at least one aroma as second gustatory agent.

According to one embodiment, the composition comprises from 0.5 to 5% by weight, for example from 1 to 2.5% by weight, of benzophenone-4 relative to the weight of the composition. According to another embodiment, the composition comprises from 1 to 3% by weight of 2-phenylbenzimidazole-5-sulfonic acid relative to the weight of the composition. In these two embodiments, from 0.3 to 0.6% by weight, more preferably from 0.4 to 0.5% by weight of sweetener is preferably used. Preferred sweeteners are sucralose or an extract of Stevia. In these two embodiments, the second gustatory agent is preferably chosen from sodium chloride and mint aromas. The sodium chloride advantageously represents from 0.2 to 0.3% by weight of the weight of the composition. The mint aroma advantageously represents from 0.05 to 0.2% by weight of the weight of the composition.

Fragrance

The composition preferably comprises odorous ingredients or fragrancing plant extracts, for example a collection of ingredients of which the INCI name appearing on the listing of ingredients of the cosmetic product proposed for sale is “Fragrance”. A fragrance is a compound or a mixture of compounds that is volatile at ambient temperature, the odour of which is detected.

The term “fragrance” is intended to mean an odorous substance or a mixture of odorous substances which evaporate. Each fragrance has what is called a top note, which is the odour that diffuses first when the fragrance is applied or when the container containing it is opened, a heart note or body which corresponds to the complete fragrance (emission for a few hours after the top note) and a base note which is the most persistent odour (emission for several hours after the heart note). The persistence of the base note corresponds to the persistence of the fragrance.

The term “fragrance” is intended to mean any organic compound capable of fragrancing the skin, the hair, the scalp, the lips or the nails.

In alcoholic products, the amount of fragrance will be more preferentially from 3 to 50% by weight, better still from 5 to 30%, even better still from 10 to 20% by weight relative to the total weight of the composition.

In care products, the amount of fragrance will be preferentially from 0.05 to 1% by weight, and better still from 0.3 to 0.7% by weight relative to the total weight of the composition.

Perfumery raw materials, aromas and mixtures thereof may be used as fragrance. They may be, independently of one another, of natural or synthetic origin.

As perfumery raw materials and aromas of natural origin, mention may, for example, be made of extracts of flowers (lavender, rose, jasmine, ylang-ylang), of stems and of leaves (patchouli, geranium, petitgrain), of fruits (coriander, aniseed, cumin, juniper), of fruit peel (bergamot, lemon, orange), of roots (angelica, celery, cardamom, iris, rattan palm), of wood (pinewood, sandalwood, gaiac wood), of herbs and grasses (tarragon, lemongrass, sage, thyme), of needles and branches (spruce, fir, pine), of resins and of balms (galbanum, elemi, benjoin, myrrh, olibanum, opopanax).

As synthetic perfumery raw materials, mention may, for example, be made of benzyl acetate, benzyl benzoate, phenoxyethyl isobutyrate, p-tert-butylcyclohexyl acetate, citronellyl acetate, citronellyl formate, geranyl acetate, linalyl acetate, dimethylbenzylcarbinyl acetate, phenylethyl acetate, linalyl benzoate, benzyl formate, ethylmethylphenyl glycinate, alkylcyclohexyl propionate, styralyl propionate and benzyl salicylate, benzyl ethyl ether, linear alkanals containing from 8 to 18 carbon atoms, citral, citronellal, citronellyloxyacetaldehyde, cyclamenaldehyde, hydroxycitronellal, lilial and bourgeonal, ionones such as alpha-isomethylionone, and methyl cedryl ketone, anethole, citronellol, eugenol, isoeugenol, geraniol, linalol, phenylethyl alcohol, terpineol and terpenes. These compounds are often in the form of a mixture of two or more of these odorous substances.

Moreover, use may also be made of essential oils, components of aromas, for instance essences of sage, of camomile, of clover, of lemon balm, of mint, of cinnamon tree leaves, of lime tree blossom, of juniper, of vetiver, of olibanum, of galbanum, of labdanum and of lavandin.

Essence of bergamot, dihydromyrcenol, lilial, lyral, citronellol, phenylethyl alcohol, alpha-hexylcinnamaldehyde, geraniol, benzylacetone, cyclamenaldehyde, linalol, ambroxane, indol, hedione, sandelice, essences of lemon, of mandarin and of orange, allylamine glycolate, cyclovertal, essence of lavandin, essence of sage, beta-damascone, essence of geranium, cyclohexyl salicylate, phenylacetic acid, geranyl acetate, benzyl acetate and rose oxide are preferably used as perfumery raw materials, alone or as a mixture.

Among the known olfactory notes, mention may, for example, be made of hesperidium fragrances, aromatics, floral fragrances, musks, fruity fragrances, spices, oriental fragrances, marine fragrances, aquatic notes, chypre fragrances, woody fragrances, green fragrances and ferns, and mixtures thereof.

According to one embodiment, the olfactory note of the fragrance of the composition is floral, for example musk floral or green floral. The note of the fragrance may be aromatic musk floral, or green rose floral.

Galenics

The composition is intended to be applied to the face and is preferably in the form of an oil-in-water or water-in-oil emulsion or an aqueous gel. The composition is, for example, in the form of a facial care cream, of a face lotion, serum or fluid, of a foundation, of a milk, of a makeup-removing lotion, of a complexion base, or of an antisun product.

According to one preferred embodiment, the cosmetic or dermatological composition is not intended to be swallowed; its administration is topical and nonoral. The composition is not a food.

According to one preferred embodiment, the invention relates to the use of rebaudioside A or of a plant extract containing it in combination with at least one salt chosen from sodium chloride, magnesium chloride and zinc chloride, and mixtures thereof, for masking the bitterness of a hydrophilic organic UV-screening agent in a manner prolonged over time. The plant extract is preferably an extract of Stevia.

According to another preferred embodiment, the invention relates to the use of sucralose in combination with at least one aroma for masking the bitterness of a hydrophilic organic UV-screening agent in a manner prolonged over time. The aroma is preferably a mint aroma.

The subject of the invention is also a cosmetic or dermatological treatment process which consists in applying a composition as described above in relation to the bitterness-masking method to at least one part of the facial skin.

Other purposes, characteristics and advantages of the invention will become clearly apparent to those skilled in the art following the reading of the explanatory description which refers to examples which are given only by way of illustration and which could not in any way limit the scope of the invention.

The examples are an integral part of the present invention and any characteristic appearing to be novel over any prior art on the basis of the description taken as a whole, including the examples, is an integral part of the invention in terms of its function and in terms of its generality.

Thus, each example has a general scope.

Furthermore, in the examples, all the percentages are given by weight, unless otherwise indicated, and the temperature is expressed in degrees Celsius unless otherwise indicated, and the pressure is atmospheric pressure unless otherwise indicated.

### Example 1

### Care Creams

The following compositions were prepared:

| | Composi- | Composi- | Composi- |
|---|---|---|---|
| | tion | tion | tion |
| INCI name or chemical name | 1 | 2 | 3 |
| Water | qs 100 | | |
| Ethylhexyl methoxycinnamate | 6.9 | | |
| Dicaprylyl carbonate | 4.3 | | |
| Glycerin | 4 | | |
| Butylene glycol | 3.7 | | |
| Butyl methoxydibenzoylmethane | 3 | | |
| Polymethyl methacrylate | 3 | | |
| Cetearyl alcohol | 2.8 | | |
| Bis-ethylhexyloxyphenol | 2 | | |
| methoxyphenyl triazine | | | |
| Behenyl alcohol | 1.7 | | |
| Pentylene glycol | 1.17 | | |
| Octocrylene | 1 | | |
| Hydrophilic UV-screening agent: | 1% | | |
| Benzophenone-4 | | | |
| Hydrophilic UV-screening agent: PSA | | 2% | |
| Hydrophilic UV-screening agent: | | | 5% |
| MBBT | | | |
| Diglycerin | 0.9 | | |
| Steareth-21 | 0.9 | | |
| Silica | 0.8 | | |
| Phenoxyethanol | 0.8 | | |
| Decyloxazolidinone | 0.8 | | |
| Sodium acrylate copolymer | 0.7 | | |
| Cetearyl glucoside | 0.7 | | |
| Hydrogenated Polyisobutene | 0.6 | | |
| Phenyl trimethicone | 0.6 | | |
| Sodium hydroxide | 0.1 | | |
| Fragrance | qs | | |
| Mixture | 100% | | |
| Addition to the above mixture of: | | | |

| | |
|---|---|
| Unipex ® mint aroma | 0.01% by weight |
| JKSS00 ® sucralose | 0.45% by weight |

PSA: Phenylbenzimidazole Sulfonic Acid
MBBT: Bis-benzotriazolyl tetramethylbutylphenol sold under the trade reference Tinosorb ® M, which is as a dispersion in a water/glycol mixture.

Gustatory and Olfactory Tests

The taste, the odour and the colour of compositions according to the invention containing a bitter hydrophilic organic UV-screening agent and gustatory agents were compared to the taste, to the odour and to the colour of reference compositions containing the same bitter hydrophilic UV-screening agent, but lacking gustatory agents. These reference tests are entitled Control 1, Control 2 and Control 3. Their composition corresponds to that of the mixture described in the previous table, containing neither mint aroma nor sucralose. The Control 3 composition contains 10% by weight of Tinosorb® M, i.e. 5% by dry weight of bis-benzotriazolyl tetramethylbutylphenol.

Make-Up of the Bitterness Panel

In order to grade the bitterness, volunteer participants were recruited for their experience in objective and descriptive characterization of sensory qualities and the ease with which they are able to disregard the hedonic components.

They were coached regularly in order to verify their reproducibility. For this, they were asked to grade the bitterness of a proposed product from 1 to 5.
5=Zero
4=Weak
3=Medium
2=Strong
1=Very strong

Each participant does not have the same sensitivity to bitterness, but must be reproducible in order to be part of the panel. A sufficient number of reproducible participants makes it possible to average the inter-individual disparities in such a way as to obtain a significant average, and an objective quantification of the bitterness.

In order to judge the reproducibility of a participant, said participant was proposed several samples blind. Among these samples two samples of the same composition were introduced. The evaluation of these two samples by each panellist should be identical. However, a difference of 1 was “tolerated” for one and the same sample.

Protocol for Sensory Analysis of Bitterness

Before beginning the grading of the bitterness, each panellist established the following conditions:
Not to have eaten or drunk anything for at least 2 hours before the test
Not to have smoked for at least 2 hours before the test
To be in the most olfactorily neutral atmosphere as possible
To avoid wearing fragranced cosmetics
To have avoided foods that are too hot and can decrease the sensitivity of the taste buds.

Before beginning the analysis, each panellist rinsed their mouth with a little water.

Each panellist took 0.2 g of product and applied it to the area around the lips (the panellist simulated a generous application to the face that would spread over onto the lips). The panellist “tasted” the product and filled-in the questionnaire.

Before proceeding with a new evaluation, the panellist rinsed their mouth with water and waited for a few minutes. The panellist then graded the intensity of the bitterness of this product in the mouth:
5=Zero
4=Weak
3=Medium
2=Strong
1=Very strong

Make-Up of the Fragrance Panel

The odour/fragrance component was judged by olfactory experts.

Results

The results are given in the three tables below.

| | Bitter- | |
|---|---|---|
| Sample evaluated | ness | Organoleptic criteria colour/odour/taste |
| Control 1 | 1.86 | |
| Control 1 | 2.57 | Colour and odour comply compared |
| + 0.225% NaCl | | with the control. |
| Control 1 | 4 | Colour and odour comply compared |
| + 0.45% Rebaten 97% ® | | with the control. |
| + 0.225% NaCl | | Bitterness greatly reduced throughout |
| | | the perception of the product in the |
| | | mouth. |
| Control 1 | 3.0 | Colour and odour comply compared |
| + 0.45% Rebaten 97% ® | | with the control. |
| | | The bitterness returns after 10 seconds |
| | | following alteration of the sweet taste. |
| Control 1 | 3.86 | Colour and odour comply compared |
| + 0.45% sucralose | | with the control. |
| JKSS00 ® | | The bitterness returns after 30 seconds |
| | | following alteration of the sweet taste. |
| Composition 1 | 4.57 | OK colour |
| | | Odour = Slight minty effect but the |
| | | distortion of the fragrancing remains |
| | | minimal, the olfactory identity is |
| | | recognisable. |
| | | The bitterness is covered throughout |
| | | the perception of the product in the |
| | | mouth. |
| Control 1 | 2.42 | OK colour |
| + 0.01% Unipex ® | | Odour = Slight minty effect but the |
| mint aroma | | distortion of the fragrancing remains |
| | | minimal, the olfactory identity is |
| | | recognisable. |
| | | The bitterness is covered throughout |
| | | the perception of the product in the |
| | | mouth. |

| | Bitter- | |
|---|---|---|
| Sample evaluated | ness | Organoleptic criteria colour/odour/taste |
| Control 2 | 2.14 | |
| Control 2 | 2.42 | OK colour and odour. |
| + 0.45% Rebaten 97% ® | | An acid effect was noted by several |
| + 0.225% NaCl | | panellists-bitterness reduced through the |
| | | perception of the product in the mouth. |
| Composition 2 | 4.7 | OK colour |
| | | Odour = slight minty effect but the |
| | | distortion of the note remains minimal, |
| | | the olfactory identity is recognisable. |
| Control 3 | 3.57 | |
| Control 3 | 4.71 | OK colour and odour |
| + 0.45% Rebaten 97% ® | | Bitterness covered throughout the |
| + 0.225% NaCl | | perception of the product in the mouth. |
| Composition 3 | 4.86 | OK colour |
| | | Odour = slight minty effect but the |
| | | distortion of the note remains minimal, |
| | | the olfactory identity is recognisable. |
| | | Bitterness covered throughout the |
| | | perception of the product in the mouth. |

Compositions 1 to 3 can be used in the cosmetics industry for application to the face and give no displeasure when they accidentally come into contact with the lips and they are capable of developing a bitterness associated with the presence of the hydrophilic organic UV-screening agent.

### Example 2

### Care Cream

The following composition was prepared.

| INCI name or chemical name | Composition 4 |
|---|---|
| Water | qs 100 |
| Ethylhexyl methoxycinnamate | 7.4 |
| Glycerin | 4.6 |
| Dicaprylyl carbonate | 4 |
| Butyl methoxydibenzoylmethane | 3 |
| Butylene glycol | 2.5 |
| Octocrylene | 2 |
| Pentylene glycol | 2 |
| Diglycerin | 1.9 |
| C₁₄-C₂₂ alcohols | 1.6 |
| Polymethyl methacrylate | 1.5 |
| Sicilia | 1.5 |
| Hydrophilic UV-screening agent: Benzophenone-4 | 1.5 |
| Steareth-21 | 1 |
| Betaine | 1 |
| Phenoxyethanol | 0.8 |
| Phenyl trimethicone | 0.7 |
| Cocoglucoside | 0.6 |
| Rosa-hybrid flower extract | 0.5 |
| C₁₂-C₂₀ Alkyl glucoside | 0.4 |
| Coconut alcohol | 0.4 |
| Polyacrylamide | 0.3 |
| C₁₃-C₁₄ Isoparaffin | 0.2 |
| Sodium hydroxide | 0.2 |
| Fragrance | qs |
| Mixture | 100% |
| Addition to the above mixture of: | |
| Extract of Stevia rebaudiana (Rebaten 97% ®) | 0.45% by weight |
| NaCl | 0.225% by weight |

Sensory Analysis

A control composition (Control 4) corresponding to composition 4 was prepared by removing the extract of Stevia and the sodium chloride. The sensory analysis conditions were created according to the protocol of Example 1. The results are given in the following table.

| Composition | Bitterness | Colour/odour |
|---|---|---|
| Control 4 | 1.14 | |
| Composition 4 | 4 | OK colour and odour |
| Control 4 | 4.57 | OK colour |
| + 0.45% sucralose | | Odour = slight minty effect but the |
| JKSS00 ® | | distortion of the note remains minimal, |
| + 0.01% Unipex ® | | the olfactory identity is recognisable. |
| mint aroma | | |

The care cream for application to the face gives no displeasure when it accidentally comes into contact with the lips and it is capable of developing a bitterness associated with the presence of benzophenone-4.

1. Method for masking the bitterness of a hydrophilic organic UV-screening agent in a cosmetic composition containing an aqueous phase and in the form of a product selected in the group consisting of a facial care cream, a face lotion, a face serum or fluid, a foundation, a milk, a makeup-removing lotion and a facial antisun product, said method comprising adding to said composition:
a sufficient amount of at least one first gustatory agent chosen from sweeteners, and
a sufficient amount of at least one second gustatory agent selected in the group consisting of taste enhancers and aromas.

2. The method of claim 1, wherein the hydrophilic organic UV-screening agent is selected in the group consisting of terephthalylidene dicamphorsulfonic acid, bis-benzoazolyl derivatives, p-aminobenzoic acid and derivatives thereof, 2-phenyl-benzimidazole-5-sulfonic acid, triethanolamine salicylate, 3-(4′-sulfobenzylidene)camphor, methylene bis-benzotriazolyl tetramethylbutylphenol, 3-(4′-trimethylammoniumbenzylidene)-1-bornan-2-one methyl sulfate, benzophenone-4, benzophenone-5, benzophenone-9, lipophilic organic UV-screening agents adsorbed or absorbed onto a hydrophilic support, and mixtures thereof.

3. The method of claim 1, wherein the organic UV-screening agent is benzophenone-4,2-phenylbenzimidazole-5-sulfonic acid, or methylene bis-benzotriazolyl tetramethylbutylphenol, or a mixture thereof.

4. The method of claim 1, wherein the hydrophilic organic UV-screening agent represents from 0.1 to 5% by weight of the weight of the composition.

5. The method of claim 1, wherein the first gustatory agent is selected in the group consisting of sucrose, glucose, fructose, acesulfame K, aspartame, cyclamic acid and also its sodium, potassium and calcium salts, isomalt, saccharin and also its sodium, potassium and calcium salts, sucralose, alitame, thaumatin, glycyrrhizic acid and its salts, neohesperidin dihydrochalcone, a steviol glucoside, neotame, the aspartame-acesulfame salt, tagatose, polyglycitol syrup, maltitol, lactitol, xylitol, erythritol and mixtures thereof.

6. The method of claim 5, wherein the steviol glucoside is chosen from rebaudioside A and plant extracts containing rebaudioside A.

7. The method of claim 5, wherein the steviol glucoside is an extract of Stevia, preferably a water-soluble extract of Stevia.

8. The method of claim 5, wherein the first gustatory agent is sucralose.

9. The method of claim 1, wherein the first gustatory agent represents from 0.1 to 1%, preferably from 0.2 to 0.6% by weight of the total weight of the composition.

10. The method of claim 1, wherein the second gustatory agent is chosen from sodium chloride and mint aromas.

11. The method of claim 1, wherein the concentration of the second gustatory agent ranges from 0.0005 to 0.5%, preferably from 0.01 to 0.3% by weight relative to the weight of the composition.

12. The method of claim 6, wherein the first gustatory agent is a steviol glucoside, and the second gustatory is chosen from sodium chloride, potassium chloride, zinc chloride and mixtures thereof.

13. The method of claim 8, wherein the first gustatory agent is sucralose and the second gustatory is one aroma.

14. The method of claim 8, wherein the second gustatory is a mint aroma.

15. The method of claim 1, wherein it is in the form of an oil-in-water emulsion.
